(19) United States
(12) Patent Application Publication
(10) Pub. No.: US 2015/0197767 A1
(43) Pub. Date: Jul. 16, 2015

(54) Spbranched1a of Solanum Pennellii and Tomato Plants with Reduced Branching Comprising this Heterologous Spbranched1a Gene

(71) Applicants: Eduardo Gonzalez-Grandio, Madrid (ES); Pilar Cubas Dominguez, Madrid (ES)

(72) Inventors: Eduardo Gonzalez-Grandio, Madrid (ES); Pilar Cubas Dominguez, Madrid (ES)

(73) Assignee: Consejo Superior de Investagaciones Cientificas, Madrid (ES)

(21) Appl. No.: 14/117,137

(22) PCT Filed: May 14, 2012
(86) PCT No.: PCT/EP12/58892

§ 371 Date: Nov. 12, 2013

(30) Foreign Application Priority Data
May 13, 2011 (EP) 11166057.7

(51) Int. Cl. C12N 15/82; C07K 14/415; A01H 5/08

(52) U.S. Cl. CPC C12N 15/8261; A01H 5/08; C07K 14/415

(57) ABSTRACT

The present invention relates to a new sequence of BRANCHED1a gene, the SpBRANCHED1a gene of Solanum pennellii, and to an expression product of said gene, to a vector, host cell, cell culture, recombinant expression system, or tomato plants comprising said SpBRANCHED1a gene. The present invention also refers to the heterologous use of the gene in tomato plants, to produce a plant having fewer and/or shorter axillary shoots than a control plant, and to a method for producing tomato plants having fewer and/or shorter axillary shoots than a control plant.

The present invention falls within the section of human necessities, in the field of agriculture; new plants or processes for obtaining them by means of biotechnology; flowering angiosperms plants, and specifically tomato plants. In particular, the present invention relates to the SpBRANCHED1a gene of Solanum pennellii, and tomato plants comprising it heterologously, wherein these tomato plants have fewer and/or shorter axillary shoots than a control plant.

### BACKGROUND ART

A central question in Biology is how the molecular evolution of genomes has led to the great variety of morphologies found in living organisms. Duplication and divergence of developmental genes is thought to have played a key role in the emergence of new traits. Genome-wide phylogenetic studies are helping us elucidate the evolutionary history of duplications in some of these gene families. However, the molecular mechanisms underlying functional divergence following duplication, and the relationship between gene evolution and emergence of morphological novelties are still not well understood.

In angiosperms, branching patterns greatly determine overall plant architecture and affect key aspects of plant life such as nutrient allocation, height, light harvesting efficiency and visibility for pollinators. Branches are generated from meristems formed in the axils of leaves (axillary meristems, AMs) after germination. AMs develop into axillary buds, structures comprising short internodes and nodes bearing leaf primordia and further AMs. Buds may remain dormant for long periods of time or undergo internode elongation to generate lateral shoots. Recent studies suggest that axillary bud development is controlled by a conserved genetic network evolved before the radiation of flowering plants. AM initiation is controlled by Lateral suppressor/MONOCULM1 genes, Blind/RAX1 genes, and LAX PANICLE genes. Auxin and strigolactone, hormones synthesised in the shoot apex and the root respectively, act as long distance signals to suppress branching. Synthesis and response to strigolactone is controlled by the More axillary branches/Ramosus pathway (MAX/RMS). Genes responding to these signals and acting inside the bud to prevent outgrowth are also conserved: maize teosinte branched1 (tb1) and its orthologs promote bud arrest in monocots (Doebley et al. 1997. Nature, 386: 485-488; Hubbard et al. 2002. Genetics, 162: 1927-1935; Hu et al. 2003. J. Plant Physiol. Mol. Biol., 29: 507-514; Kebrom et al. 2006. Plant Physiol, 140: 1109-1117; Takeda et al. 2003. Plant J., 33: 513-520). In dicots, duplications of an ancestral tb1-like gene generated the CYC/TB1 clade comprising three types of genes (Howarth and Donoghue 2006. J. Plant Physiol. Mol. Biol., 29: 507-514). One of them includes BRANCHED1 (BRC1) which has retained most of the branch-suppressing activity in Arabidopsis (Aguilar-Martinez et al. 2007. Plant Cell, 19: 458-472). tb1/BRC1 genes encode transcription factors of the TCP family (Cubes et al. 1999. Plant J., 18: 215-222). TCP transcription factors are involved in the control of cell growth and proliferation in meristems and lateral organs, and they have played a key role in the evolution of innovations such as flower zygomorphy.

Despite the general conservation of genes and pathways, a great diversity of branching patterns is found in angiosperms. Moreover, timing of AM initiation, and branch outgrowth in response to environmental and endogenous signals are very divergent among clades. Molecular evolution of genes controlling branch outgrowth must have played an important role in the generation of this diversity. Putative candidate genes to be targets for selection during the evolution of new branching patterns are tb1/BRC1-like genes. Indeed, in maize, artificial selection of tb1 over-expressing alleles was responsible for the strong apical dominance of the domesticated maize plants (Doebley, Stec and Hubbard 1997, Wang et al. 1999. Nature, 386: 485-488).

BRC1-like genes from Solanum lycopersicum (Solanaceae, Asteridae), a dicot species distantly related to Arabidopsis (Brassicaceae, Rosidae) and with diverging branching patterns. In contrast to Arabidopsis, which has an indeterminate (monopodial) growth habit, tomato, has a determinate (sympodial) growth habit: while in Arabidopsis the shoot apical meristem (SAM) grows indefinitely, in tomato, after the production of 8-12 leaves, the SAM is terminated in a cymose inflorescence. Upon flowering, the lateral meristem in the uppermost leaf axil (sympodial bud) is immediately released to give a shoot. This lateral shoot generates several leaves before terminating with another inflorescence, and so on. Additional lateral shoots may grow out from buds of the primary shoot.

In the international patent application WO 2010/081917 A1 was described the sequence of SIBRC1 from Solanum lycopersicum and inventors elucidated, by means of RNAi experiments, that the SIBRC1b gene plays a more important role in the control of axillary bud development and branch outgrowth than SIBRC1a. Furthermore, this patent application related to the promoter of said gene. Despite the results shown in that document, it remains in the state of the art the problem to produce plants that have effectively a substantial reduction in the number of branches in order obtain plants with better fruits and also to improve the mechanical harvesting of these fruits.

### SUMMARY OF THE INVENTION

The invention relates to a new sequence of BRANCHED1a gene, the SpBRANCHED1a gene of Solanum pennellii, and to an expression product of said gene, to a vector, host cell, cell culture, recombinant expression system, or tomato plants comprising said SpBRANCHED1a gene. The present invention also refers to the heterologous use of the gene in tomato plants, to produce a plant having fewer and/or shorter axillary shoots than a control plant, and to a method for producing tomato plants having fewer and/or shorter axillary shoots than a control plant.

In the present invention the term SpBRANCHED1a as synonymous of SpBRC1a can be used. Along the present invention, to refer to this gene can be used the term “isolated polynucleotide” or “polynucleotide” encoding a polypeptide comprising the amino acidic sequence of the protein SpBRANCHED1a (SpBRC1a) from Solanum pennellii, taking into account that, by alternative splicing, two proteins are translated from a single genomic sequence, a short and a long protein, differing in their C-terminal domain.

As it is known in the state of the art, in the case of tomato BRC1-like genes, SIBRC1b seems to have retained the ancestral BRC1-like gene function in the suppression of shoot branching while inventors have not been able to establish any relevant role in this process for SIBRC1a. Consistently, an asymmetrical distribution of selective constraints has been detected in the evolution of Solanum BRC1a and BRC1b lineages: the coding sequence of BRC1b genes have evolved under a strong purifying selection while the BRC1a lineage had experienced a decrease of evolutionary constraints. Therefore, despite this evidence, the present invention shows that SpBRC1a plays an unexpected role in suppressing shoot branching when is expressed heterologously in tomato, in opposition with the non-contributing role to this branchless phenotype of the native gen SIBRC1a.

The distinguishing features of the present invention in respect to the relevant background art is the new sequence SpBRC1a from Solanum pennellii and its use to produce plants which express SpBRC1a heterologously in tomato plants, as for example, in Solanum lycopersicum plants. The technical effect resulting from the distinguishing features is a stronger apical dominance, an improved architecture and more reduced number of lateral branches (branchless phenotype) than control plants, M82 tomato plants. This technical effect implies an improvement of the described phenotype with respect to control plants, described in the examples of the present invention (e.g. IL3-5 plants). This improvement is due to the functional substitution of the native gene SIBRC1a from Solanum lycopersicum. Therefore, the expression of SpBRC1a in Solanum lycopersicum implies an improvement of branchless phenotype in tomato plants.

Then, the objective technical problem solved by the present invention is the production of tomato plants with the maximal reduction in number and length of axillary shoots as possible because lateral shoot branching is an undesired trait in the domesticated tomato as it diverts assimilates away from developing fruits.

In conclusion, the present invention provides new plants with branchless phenotype modifying or adapting the closest prior art to provide the described technical effect. Therefore, the present invention provides tools to the state of the art to improve the branchless phenotype in tomato plants.

It must be stressed that the present invention emphasizes an unexpected or surprising effect: Since is not SIBRC1a but SIBRC1b the sole responsible in controlling the shoot branching in Solanum lycopersicum plants, the native SIBRC1a gene does not play a major role in the suppression of axillary bud outgrowth in these tomato plants. In the state of the art is not suggested that any homologous gene of SIBRC1a in any tomato plant would have a different activity/function than it is demonstrate with SIBRC1a, despite the high identity between proteins SpBRC1a and SIBRC1a, being 92-93% identical (table 1), the role of SpBRC1 is very different when expressed in Solanum Lycopersicum than the role of the native protein SIBRC1a.

The plants of the present invention also have the advantage to facilitate the pruning and more important than this, because the plants of the invention don't need the pruning of lateral shoots, fewer wounds can be practice and then, potentially, a less number of pathogens will infect the plant.

The present invention is susceptible of industrial application, i.e. for avoid the deviation of assimilates away from developing fruits and also to improve the mechanical harvesting of tomatoes.

Thus, a first aspect of the present invention refers to an isolated polynucleotide, encoding a polypeptide comprising:
a. an amino acidic sequence from Solanum pennellii which is at least 90% identical to SEQ ID NO: 1 or which is at least 90% identical to SEQ ID NO: 2, or
b. the amino acidic sequence from Solanum pennellii SEQ ID NO: 1 or SEQ ID NO: 2.

In the present invention, the terms “polypeptide” and “amino acidic sequence” are synonymous.

The amino acidic sequence from Solanum pennellii which is at least 90% identical to SEQ ID NO: 1 or which is at least 90% identical to SEQ ID NO: 2 are isoforms of these sequences or sequences with a maximum of 10% of changes in amino acids between them sequences having substantially the same activity/role in the branchless phenotype than SEQ ID NO: 1 or SEQ ID NO: 2. Therefore, the first aspect of the present invention also refers to an isolated polynucleotide, encoding a polypeptide comprising:
a. the amino acidic sequence of any protein SpBRC1a from Solanum pennellii, wherein the protein SpBRC1a has substantially the same role in the branchless phenotype than SEQ ID NO: 1 and/or SEQ ID NO: 2 in tomato plants, or
b. the amino acidic sequence from Solanum pennellii SEQ ID NO: 1 or SEQ ID NO: 2.

SEQ ID NO: 1 or SEQ ID NO: 2 are the two resulting sequences of alternative splicing of the gene SpBRC1a, coded in the genomic sequence (example 1). Since the putative splicing sites (gt/ag) of intron I were found in all the Solanum BRC1a genes studied but not in BRC1b genes, inventors demonstrate that this pattern of alternative splicing of BRC1a gene was maintained in respect to SpBRC1a gene identifying said intron I in the genomic sequence of SpBRC1a gene. Then, these two sequences SEQ ID NO: 1 and SEQ ID NO: 2 are involved in the branchless phenotype. The two sequences have different C-terminal (C-t) domains due to a frameshift caused by the alternative splicing of intron I, like in SIBRC1a gene.

A preferred embodiment refers to the isolated polynucleotide wherein said polynucleotide is SEQ ID NO: 3 encodes SEQ ID NO: 1, or SEQ ID NO: 4 encodes SEQ ID NO: 2. The sequence SEQ ID NO: 3 is the Coding Sequence (CDS) of the short variant of SpBRC1a protein; SEQ ID NO: 1. The sequence SEQ ID NO: 4 is the CDS of the long variant of SpBRC1a protein; SEQ ID NO: 2.

In another preferred embodiment of the present invention, the isolated polynucleotide is the genomic sequence SEQ ID NO: 5. This sequence has the introns described for SIBRC1a.

The term “% identical” as is understood in this invention refers to the % of identity between two amino acid sequences. The % of identity is a count of the number of positions over the length of the alignment of two sequences where all of the amino acids at that position are identical.

Preferably, the amino acidic sequence from Solanum pennellii which is at least 90% identical to SEQ ID NO: 1 or to SEQ ID NO: 2 can be at least 91%, 92%, 93%, 94%, 95%, 96%, 97%, 98% or 99% identical to SEQ ID NO: 1 or to SEQ ID NO: 2. Furthermore, the amino acidic sequence from Solanum pennellii which is at least 90% identical to SEQ ID NO: 1 or which is at least 90% identical to SEQ ID NO: 2 can be an amino acidic sequence with at least less than 90% identical to SEQ ID NO: 1 or SEQ ID NO: 2 if said amino acidic sequence has substantially the same role in the branchless phenotype of SEQ ID NO: 1 and/or SEQ ID NO: 2 in tomato plants, and also is a sequence from Solanum pennellii.

In order to yield information about of the identity of different sequences of BRC1a proteins in tomato plants, table 1 and table 2 is presented.

TABLE 1
Percentage of identity of several homologous amino acidic sequences
respect to SEQ ID NO: 1 or SEQ ID NO: 2 from different tomato plants.

| | | | Size | |
|---|---|---|---|---|
| | Size (amino | | (amino | Identity |
| Species 1 | acids) | Species 2 | acids) | (%) |
| S. pennellii | 313 | S. lycopersicum | 325 | 92 |
| Short (SEQ ID | | Short (SEQ ID | | |
| NO: 1) | | NO: 8) | | |
| | | S. tuberosum | 300 | 85 |
| | | Short (SEQ ID | | |
| | | NO: 9) | | |
| S. pennellii | 349 | S. lycopersicum | 346 | 93 |
| Long (SEQ ID | | Long (SEQ ID NO: | | |
| NO: 2) | | 10) | | |
| | | S. tuberosum | 336 | 86 |
| | | Long (SEQ ID NO: | | |
| | | 11) | | |

The methods to compare the sequences obtaining identity percentages are known in the state of the art, and include, but not limited to, the program GAG, including GAP (Devereux et al. 1984. Nucleic Acids Research, 12: 287), Genetics Computer Group University of Wisconsin, Madison, (Wis.); BLAST, BLASTP, ClustallW2 or Phylemon web server (Tarraga et al. 2007. Nucleic Acids Res, 35: W38-42). The percentage of identity has been chosen according to the ClustallW2 Multiple Sequence Alignment from European Bioinformatics Institute.

As it is shown in table 1, despite the high identity between proteins SpBRC1a and SpBRC1a, being 92-93% identical, the role of SpBRC1a very different, as demonstrated in the present invention, when expressed in Solanum Lycopersicum compared with the role of the native protein SIBRC1a.

TABLE 2
Percentage of identity between SEQ ID NO: 1, SEQ ID NO: 2
(SpBRC1a) and SpBRC1b from Solanum pennellii tomato plants.

| | Size (amino | | Size (amino | Identity |
|---|---|---|---|---|
| Species 1 | acids) | Species 2 | acids) | (%) |
| S. pennellii | 313 | S. pennellii | 349 | 92 |
| Short (SEQ ID | | Long (SEQ ID | | |
| NO: 1) | | NO: 2) | | |
| S. pennellii | 313 | S. pennellii | 362 | 37 |
| Short (SEQ ID | | SpBRC1b | | |
| NO: 1) | | (SEQ ID NO: | | |
| | | 10) | | |
| S. pennellii | 349 | S. pennellii | 362 | 42 |
| Long (SEQ ID | | SpBRC1b | | |
| NO: 2) | | (SEQ ID NO: | | |
| | | 10) | | |

A preferred embodiment of the present invention refers to the polynucleotide operatively linked to any regulatory sequence that controls the expression of said polynucleotide. In further preferred embodiment of the present invention, said regulatory sequence is SEQ ID NO: 6, SEQ ID NO: 7 or CaMV35S.

The term “regulatory sequence that controls the expression of a polynucleotide” refers to a nucleotidic sequence having an effect on the functionality of the polynucleotide gene referred to the beginning of the transcript from a DNA sequence or at the start of translation of a sequence of RNA or other sequences not described. By way of example, between regulatory sequences of the gene expression referred to in the present invention are the promoters and other less common as certain introns. The nature of these regulatory sequences differs depending on the host organism; in prokaryotes, these sequences of control usually include a promoter, a site of union ribosomal, and signs of completion; in eukaryotes, usually, these sequences of control include promoters, signs of termination, intensifiers and sometimes silencers.

As it is used here, the term “promoter” refers to a region of the DNA upstream from the start point of the transcription of a gene, and particularly, that is able to start the transcription in a plant cell. Examples of promoters include but are not limited to, promoters obtained from plants, virus of plants or bacteria that can express genes in cells of plants, as Agrobacterium or Rhizobium. Promoters can be classified, for instance, as inducible and constitutive promoters. The sequence SEQ ID NO: 6 is the inducible promoter of the gene SIBRC1a from Solanum lycopersicum. The sequence SEQ ID NO: 7 is the inducible promoter of the gene SIBRC1a from Solanum tuberosum.

The term “operatively linked” refers to a juxtaposition in which the components as well described have a relationship that enables them to work in the intentional way. A polynucleotide operatively linked to any regulatory sequence that controls the expression of said polynucleotide is linked in such a way that the expression of the coding sequence of the polynucleotide is achieved under conditions compatibles with the regulatory sequence.

To refer to any of the polynucleotide described in the above aspect and its preferred embodiments of the present invention, the term “polynucleotide of the invention” or “polynucleotide of the present invention” can be used.

Another aspect of the present invention refers to an expression product of the polynucleotide of the invention. To refer to any of the expression product of the polynucleotide of the present invention, the term “expression product of the invention” or “expression product of the present invention” can be used.

The term “product of the expression of the sequence” as it is used in the present invention refers to any product derived from the expression of the polynucleotide of the invention. Thus, as product derived from the expression of the polynucleotide of the invention is understood, for example, the RNA that is obtained from the transcription of said polynucleotide, the processed RNA, the resulting protein in the translation of RNA, or subsequent modifications to the polynucleotide inside the cell, provided that the resulting sequence of these modified polynucleotides having its origin in the original sequence or the functional role described in the present invention.

A further aspect of the present invention refers to an expression vector comprising the polynucleotide of the invention. To refer to any vector comprising the polynucleotide of the present invention, the term “vector of the invention” or “vector of the present invention” can be used.

The term “vector” refers to a DNA fragment that has the ability to replicate in a given host and, as the term indicates, it can serve as a vehicle to multiply another fragment of DNA that has being linked to the same (insert). The term “insert” refers to a DNA fragment that is linked to the vector; in the case of the present invention, the vector comprises any of the polynucleotides of the invention that can be replicated in the appropriate host. The vectors may be plasmids, cosmids, bacteriophages or viral vectors, without excluding another type of vectors that correspond to this definition of vector.

Another aspect of the present invention refers to a host cell comprising the polynucleotide, the expression product, or the vector of the invention, wherein if the host cell is a plant cell, said plant cell is not from the species Solanum pennellii. That is to say, the polynucleotide of the present invention (the mean core corresponding to the gen of Solanum pennellii) is present in said host cell in heterologous form. A preferred embodiment refers to the host cell of the present invention, wherein said cell is a plant cell. More preferably the plant cell is a Solanum lycopersicum cell.

The term “host cell” as it is used in the present invention relates to any procariotic or eukaryotic cell, essentially, it refers to a eukaryotic plant cell and within this group, more preferably, those cells belonging to the Kingdom Plantae, wherein any of theses cells comprises polynucleotide, the expression product, or the vector of the invention. Thus, the term plant cell comprises at least one cell of the parenchyma, meristematic cell or of any kind of plant cell, differentiated or undifferentiated. Preferably the plant cell is a tomato plant cell and more preferably is a Solanum lycopersicum cell.

To refer to any host cell comprising the polynucleotide of this invention, the term “host cell of the invention” or “host cell of the present invention” can be used.

The present invention further relates to a cell culture comprising the host cell of the invention. To refer to any cell culture comprising the host cell of the present invention, the term “cell culture of the invention” or “cell culture of the present invention” can be used.

The term “cell culture” refers to a cultivation of cells isolated from the same or different type of tissue, or a collection of these cells organized in parts of a plant or in tissues (tissue culture). Types of this kind of cultures are, e.g. but without any limitation, a culture of protoplasts, calli (groups of plant cells undifferentiated able to regenerate a complete plant with the appropriate organogenic program) or a culture of plant cells that are isolated from plants or parts of plants such as embryos, meristematic cells, pollen, leaves or anthers.

Another aspect of the present invention refers to a recombinant expression system comprising the polynucleotide, the expression product, the vector, or the host cell of the invention.

The recombinant expression system can be, but without limitation, e.g. a recombinant host cell or a recombinant bacteriophage or their combination with any helper virus. Recombinant manufacturing involves the expression of a DNA construct encoding for the desired protein in a recombinant host cell. The host cell can be either prokaryotic or eukaryotic. Recombinant manufacturing, however, does have its difficulties. Expression constructs must be optimized for a particular protein and for a particular host cell. Expressing a recombinant protein in a host cell exposes the recombinant protein to a new set of host cell enzymes, such as proteases, which can modify or even degrade the recombinant protein. Modification and degradation of the recombinant protein of interest is undesirable.

A further aspect of the present invention refers to a use of the polynucleotide, the expression product, the vector, the host cell, the cell culture, or the recombinant expression system of the invention, to produce a tomato plant, but not a plant of the species Solanum pennellii, having fewer and/or shorter axillary shoots than a control plant.

The term “axillary shoots” can be used as synonymous of “lateral branches”, “lateral shoots”, “secondary shoots”, “axillary shoot branching”, “axillary bud outgrowth”, and refers to a shoot growing from the axillary bud located between the leaf and the stem, that is to say, growing in a node. In a control plant, the stem displays branching due the outgrowth of axillary buds in the individual node positions.

In the tomato plants of the present invention, the length of the basal lateral branches (nodes 1-6) presents significant differences with regard to the length of the lateral branches in the control.

In the present invention, the term “fewer and/or shorter” relates to significant differences between the presence/absence and/or the length of axillary shoots, respectively, in the equivalent node of the plants of the invention and control plants. The skilled person can arrive to these significant differences using any statistical and any method known in the background art. Then, in the present invention plants having fewer and/or shorter axillary shoots than a control plant have branchless phenotype.

For instance, as shown in the examples of the present invention, IL3-5 plants had significantly fewer and/or shorter axillary shoots than M82 plants in cotyledons, and nodes 1, 4, 5 and 6.

The control plant is any tomato plant, wild type carrying empty vectors, but in any case the control plant does not contain the polynucleotide of the present invention. Preferably the control plant is a tomato plant belonging to the species Solanum lycopersicum, preferably the control plant is a M82 cultivar of tomato. The control plant has the same taxonomic category than the tomato plant comprising the polynucleotide of the invention in other to compare the number or/and length of lateral branches.

Another aspect of the present invention refers to a tomato plant, but not a Solanum pennellii plant, comprising the polynucleotide, the expression product, the vector, the host cell, the cell culture, or the recombinant expression system of the invention, wherein said plant has fewer and/or shorter axillary shoots than a control plant. Thus, the polynucleotide of the present invention (the mean core corresponding to the gen of Solanum pennellii) is present in plant in heterologous form. Preferably the tomato plant belongs to the species Solanum lycopersicum.

The tomato plant of the present invention can be selected from potentially tomato plant species, for instance the tomato plant can be selected from tomato plants with red, yellow, orange or yellow-green fruit, and without limitation to theses species: Solanum lycopersicum, Solanum peruvianum, Solanum pimpinellifolium, Solanum lycopersicoides, Solanum sitiens, Solanum juglandifolium, Solanum ochranthum, Solanum cheesmaniae or Solanum galapagense.

Preferably, the tomato plant belongs to the species Solanum lycopersicum. More preferably the variety of the tomato plant can be selected from the list of stock names in the SOL Genomics network (SGN) Breeders Toolbox (http://solgenomics.net/breeders/index.pl). Preferably the variety of the tomato plant from the species Solanum lycopersicum can be selected from the list, but without limitation, Anna russian, Applause, Aussie, Baladre, Bella rosa, Black cherry, Black russian, Blondkopfchen, Brandywine, Carbon, Ceylan, Cherokee purple, Cherry yellow pear, Black Plum, Comanche, Copy, Costoluto genovese, Ditmarcher, Eros, Gallician, Glacier, Gartenperle, Green sausage, Grushovka, Harzfeuer, Hugh, Japanesse black, Jersey devil, Kosovo, Krim black, Kumato, Liguria, Limachino, Lime green salad, Manitoba, Marvel stripe, Moneymaker, Muchamiel, Opalka, RAF, Black Pear, Hawaian pineapple, Rio grande, San marzano, Siberian, Sprite, Sugary, Sun sugar, Tigerella, Valencian, White Queen or Window box Roma.

A further aspect of the present invention refers to a tomato plant comprising, heterologously, a fragment of the chromosome III of Solanum pennellii that comprises a polynucleotide encoding a polypeptide comprising:

a. an amino acidic sequence from Solanum pennellii which is at least 90% identical to SEQ ID NO: 1 or which is at least 90% identical to SEQ ID NO: 2, or
b. the amino acidic sequence from Solanum pennellii SEQ ID NO: 1 or SEQ ID NO: 2.
wherein said plant has fewer and/or shorter axillary shoots than a control plant

A preferred embodiment of the invention refers to said tomato plant comprising, heterologously, a fragment of the chromosome III of Solanum pennellii that comprises a polynucleotide encoding a polypeptide, wherein said fragment of the chromosome III corresponds to the fragment comprised between the DNA marker TG284A and the DNA marker TG244 (FIG. 2). Preferably the fragment of the chromosome III corresponds to the fragment comprised between the DNA marker T0794 and the DNA marker TG244; or more preferably to the fragment comprised between the DNA marker TG377 (or the marker TG126 or CD4A) and the DNA marker TG244. The FIG. 2 shows all the proposed DNA markers within chromosome III of Solanum pennellii. The cited markers can be analyzed in detail in the Sol Genomics Network (SGN) database.

A preferred embodiment of the invention refers to the plant comprising, heterologously, a fragment of the chromosome III of Solanum pennellii, wherein comprises a polynucleotide encoding a polypeptide comprising SEQ ID NO: 3 encoding SEQ ID NO: 1, or SEQ ID NO: 4 encoding SEQ ID NO: 2. Preferably said polynucleotide is the genomic sequence SEQ ID NO: 5.

A preferred embodiment of this invention refers to any plant described above wherein the expression of the polynucleotide of the invention is greater than the expression of the homologous native gene BRC1a.

The plant contains the polynucleotide of the invention in homozygosis, heterozygosis or hemicigosis. A further embodiment of the present invention refers to any plant described above, wherein the polynucleotide is integrated in their genome, preferably integrated in homozygosis

Another aspect refers to a germplasm of the plant of the present invention. Preferably the germplasm is a seed or pollen.

The term “plant of the present invention” includes each of the parties on said plant, which can be conserved or cultivated in isolation or in combination, as well as the germplasm. The germplasm is defined by the biological material that contains the intraspecific genetic variability or by the genetic materials that can perpetuate a species or a population of said plant. Thus germplasm is the seed, tissue culture for any part of the plant or plants established in ex situ collections, without excluding any other material in the scope of this definition.

The pollen has high level of interest since the transmission of the genetic and phenotypic characters can be carried out by the pollination of any plant variety compatible with the pollen that is referenced. In this way can be produce a plant which includes the polynucleotide of the invention, after the respective cross and selection, it can be obtained a plant in which the polynucleotide integrates a suitable number of copies in stable condition in order to obtain the same desirable branchless phenotype in the subsequent generations.

To refer to any of the tomato plants of the present invention, the term “plant of the invention” or “plant of the present invention” can be used.

The plant of the invention does not belongs to the species Solanum pennellii, then being the polynucleotide of the present invention expressed in different plant species that Solanum pennellii, therefore, the polynucleotide is expressed heterologously.

A further aspect of the present invention refers to a method for producing tomato plants having fewer and/or shorter axillary shoots than a control plant, comprising:
a. Transforming at least a tomato plant cell with a heterologous polynucleotide comprising:
   i. an amino acidic sequence from Solanum pennellii which is at least 90% identical to SEQ ID NO: 1 or to SEQ ID NO: 2, or
   ii. the amino acidic sequence from Solanum pennellii SEQ ID NO: 1 or SEQ ID NO: 2, and
b. growing the plant cell obtained in the step (a) in a suitable medium to produce at least a plant which expresses the heterologous polynucleotide.

Another embodiment of the present invention refers to the method for producing tomato plants having fewer and/or shorter axillary shoots than a control plant, wherein said tomato plant belongs to the species Solanum lycopersicum.

The plant of the invention can be achieved by genetic transformation mediated by biolistic, Agrobacterium tumefaciens or any other technique known by the skilled in the art (e.g. transformation of protoplasts), that will allow the integration of the polynucleotide of the invention in any of the DNA of the plant; genomic, chloroplastic or the polynucleotide of the invention by crossing and selection.

The suitable medium growing the plant cell obtained in the step (a) is a known medium by the skilled in the art.

The production of a plant as is indicate in step (b) can be performed by means techniques known by the skilled in the art, for instance:
The cultivation of embryos: Isolation of zygotic embryos promoting their growth as plant in an artificial environment
Somatic embryogenesis: Production of embryos from somatic tissues, such as microspores or leaves
Organogenesis: Production of organs such as stems or roots from various tissues of the plant.

The plant of the present invention can be obtained according with other microbiologic processes as for example:
Obtaining cybrids: It is produced a cell with its cytoplasm and the cytoplasm of the other cell and this cell can be grown in a suitable medium to produce a plant which expresses the heterologous polynucleotide.
Fusion of somatic cells (preferably protoplast fusion): at the cytoplasmic level hybrid plants can be the result of: (a) The sum of the cytoplasm of both parental; (b) The cytoplasm of a single parental; (c) a cytoplasm hybrid result of recombination of the genomes extranuclears of both cells. The fusion of somatic cells can be applied; to overcome the incompatibility in interspecific crosses, or a better utilization of the interspecies variation and extraspecific in interspecific compatible crosses.

Unless otherwise defined, all technical and scientific terms used herein have the same meaning as commonly understood by one of ordinary skill in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples, figures and sequence listing are provided by way of illustration and are not intended to be limiting of the present invention.

### BRIEF DESCRIPTION OF THE FIGURES

In order to complement the description that is being made and in order to help better understanding of the characteristics of the invention and in agreement with some preferred examples, figures have been included where, with illustrative and non-limiting character, the following is represented:

FIG. 1. Shows the BRC1-like genes and proteins in tomato.

(a) Maximum Likelihood (ML) phylogenetic tree with 1000 bootstrap pseudoreplicates of class II TB1/CYC genes from tomato (SI, bold) and representative class II TCP members from Antirrhinum majus (Am), Arabidopsis thaliana (At), Solanum tuberosum (St) and Populus trichocarpa (Pt). Branches with support of 500 or more are indicated. AtTCP4/CIN are the outgroups. (b) Gene structure of SIBRC1a and SIBRC1b. Coding sequences are shaded in black, introns in white, 5′ UTR and 3′UTR in light grey. Below each gene, predicted proteins encoded by the isolated cDNAs are represented. Blue boxes indicate TCP and R domains. Red boxes indicate the alternative coding frame in the short SIBRC1a protein. Dark grey box indicates the alternative intron translated in the long protein.

FIG. 2. Shows the map of chromosome III of Solanum lycopersicum (a) and Solanum pennellii (b) and the relative position of BRC1 gene.

In both maps are indicated several DNA markers and their relative position between them and between BCR1a gene and IL3-5 introgressed fragment.

TG479, T1286, CT171, T1511, T0794, TG244 are DNA markers from chromosome III of Solanum lycopersicum and TG40, CAB3, TG13S, CT31, TG114, TG585, TGS17, TG130, TG13B, CT22, TG66, TG39, TG247, TG288, TG50C, TG102, CT90A, TM8, TGS99, TG246, TG457, TG42, TG134, TG284A, TG377, CD4A, TG126, TG152, CD71, TG406B, TG214, CD69, CT250, TG244 and TG94. The fragment of the Chromosome III from Solanum Pennellii introgressed in Solanum lycopersicum in IL3-5 introgression line is shown with the bar IL 3-5. (a) and (b) shows some common DNA markers in both chromosomes.

FIG. 3. Shows the Secondary structure prediction of the long (top) and short (bottom) C-t motifs of SIBRC1a according to Jpred.

E, extended secondary structure, H, α-helix. Jnet, solvent accessibility predictions. B, buried, -, exposed. Jnet Rel, reliability of prediction accuracy, ranges from 0 to 9.

FIG. 4. Shows the analysis of SIBRC1a function.

(a) Shoot branching phenotype of Slbrca-v171i point mutants compared to their wild type siblings. Lateral branch length of mutant tomato plants and their wild type siblings at anthesis of the first flower. Standard errors (±s.e.m.) are represented. N_{brc1a-v171i}=7, N_{wt}=25. (b) Shoot branching phenotype of IL3-5 and M82 plants measured as in (a) N_{IL3-5}=12, N_{wt M82}=15. Asterisks indicate significant differences (p<0.05). (c) Young M82 wild type (left) and IL3-5 (right) plants grown in the same conditions. Arrows indicate branches grown out in wild type plants and arrested buds in IL3-5. (d). Relative SIBRC1a mRNA levels in axillary buds of M82 and IL3-5 plants. The asterisk indicates a significant difference (p<0.05).

FIG. 5. Shows the phenotype of adult M82 and IL3-5 plants after flowering.

### EXAMPLES

The following examples provide a description, of illustrative and non-limiting character, of some of the assays and operating conditions claimed in the list given below that refers to the cloning and use SpBRC1a as a suppressor of shoot branching in tomato, as well as plants of Solanum lycopersicum expressing the SpBRC1a gen.

In the present examples the term SpBRC1a is equivalent to an isolated polynucleotide encoding a polypeptide comprising the amino acidic sequence of SEQ ID NO: 1 (short splicing alternative protein) or SEQ ID NO: 2 (long splicing alternative protein). SIBRC1a and SIBRC1b refer to the gen BRANCHED1a or BRANCHED1b of Solanum lycopersicum.

### Example 1

### Identification of Tomato BRC1-Like Genes

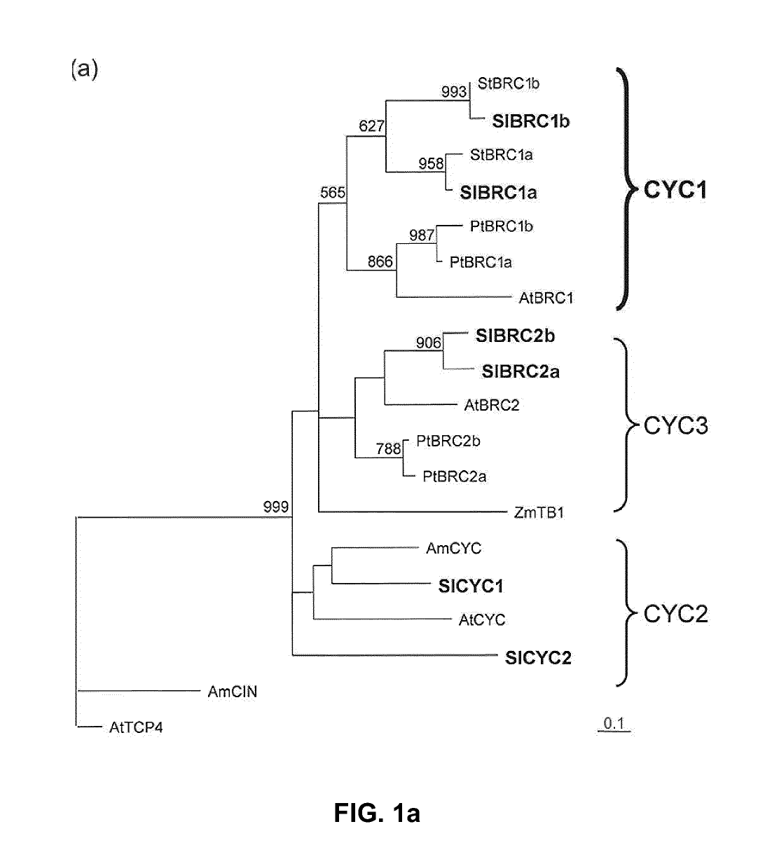

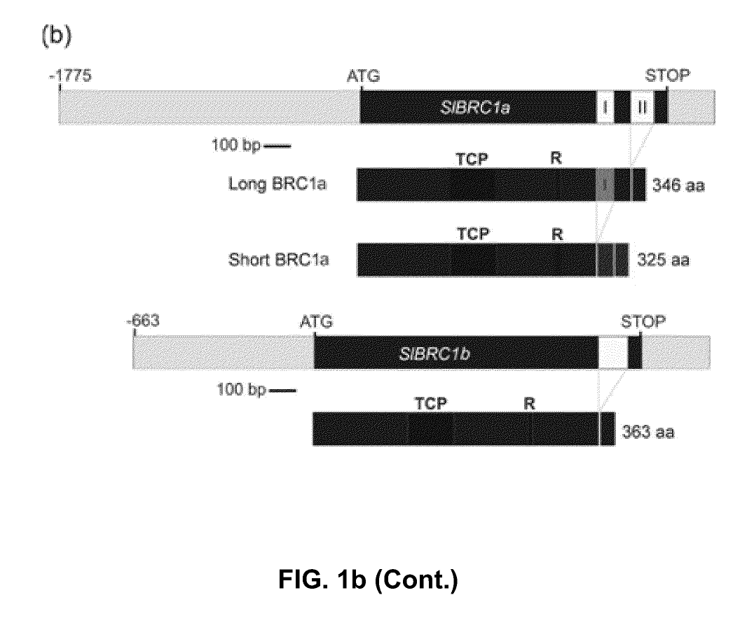

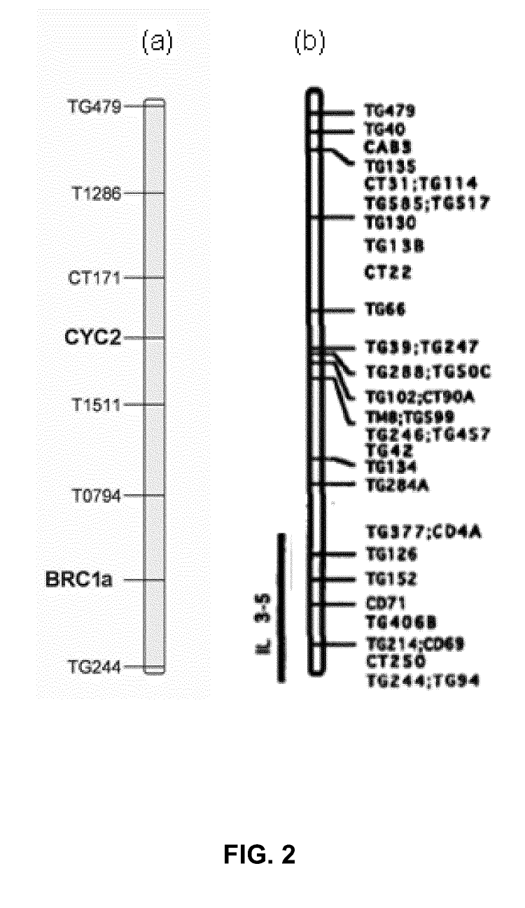

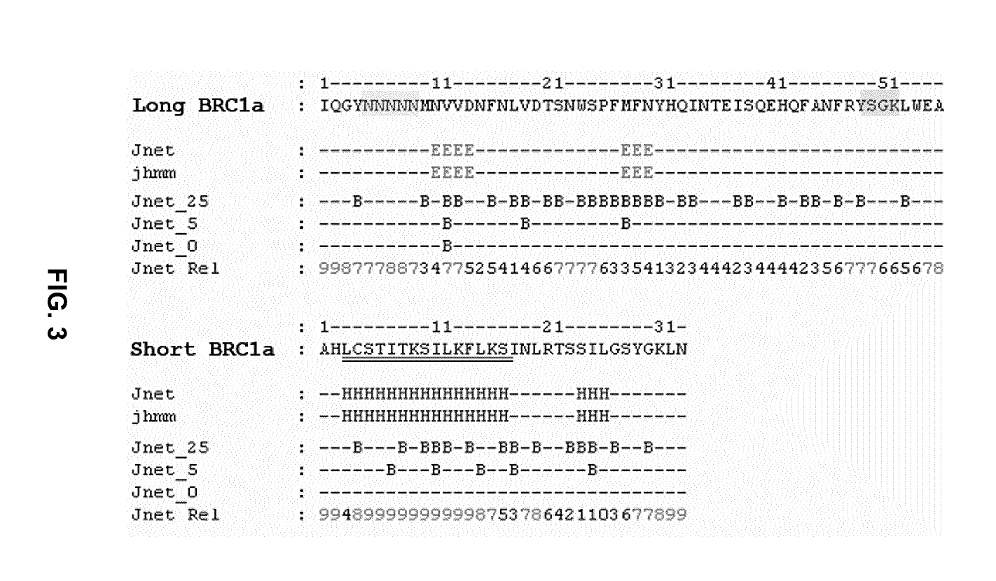

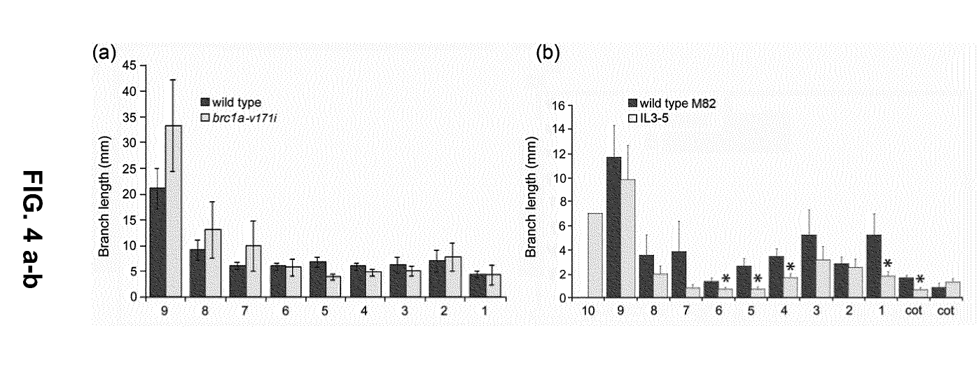

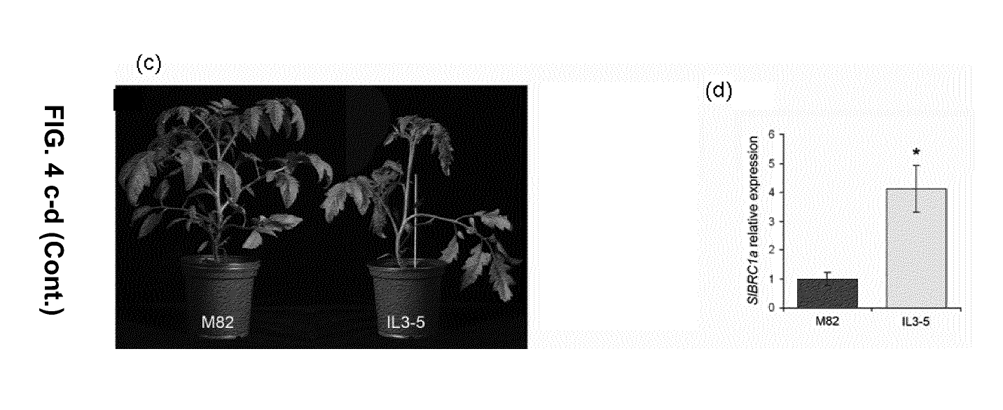

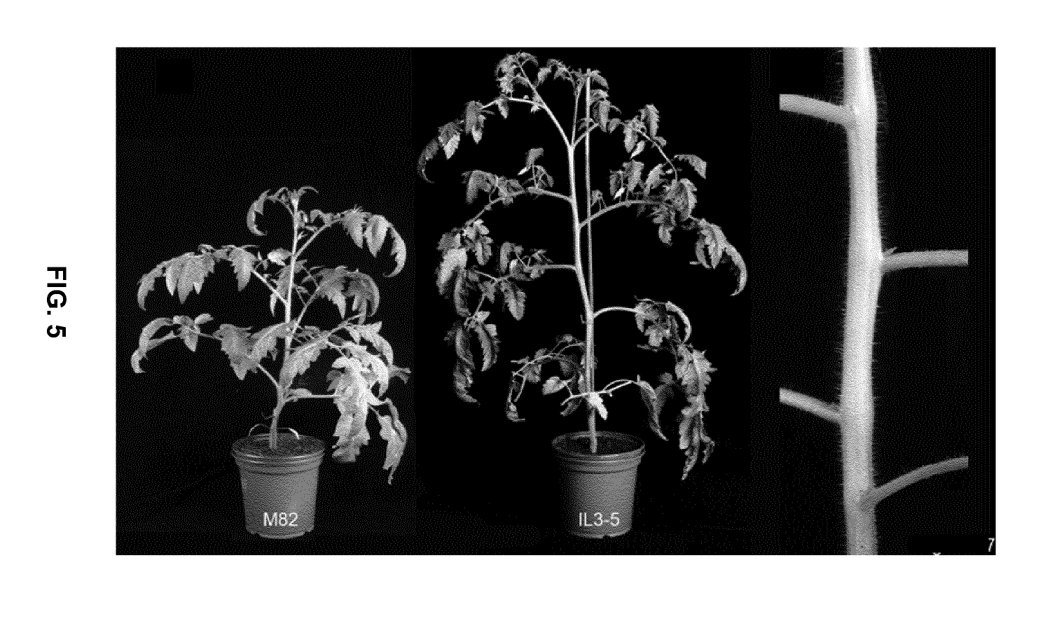

The inventors searched for CYC/TB1 type genes in tomato genomic and EST databases (Mueller 2009. The Plant Genome, 2: 78-92) and identified six TCP genes of this group. Phylogenetic analyses showed that two were CYCLOIDEA (CYC)-like genes, two were BRC2-like genes and another two were BRC1-like genes. These six TCP genes were named SICYC1, SICYC2, SIBRC2a, SIBRC2b, SIBRC1a and SIBRC1b (Solanum lycopersicum CYC, BRC2, BRC1) respectively (FIG. 1a) and were mapped to tomato chromosomes 2-6 (FIG. 2). CYC-like genes have been shown to be expressed in floral meristems and to be mainly involved in the control of flower shape in many species. Inventors focussed on the two BRC1-like genes (SIBRC1a and SIBRC1b) and investigated their roles during tomato lateral shoot development.

Inventors first studied the transcriptional activity of these genes. For SIBRC1a, inventors isolated two cDNAs generated by alternative splicing: a short one, with a 978 base pairs (bp) long open reading frame (ORF) split by two introns (introns I and II), and a long one (1041 bp), in which intron I was retained and translated (FIG. 1b). Splicing of intron I was a relatively rare event as long cDNAs were isolated 9 times more frequently than short cDNAs. These cDNAs encoded two proteins with identical N-terminal, TCP and R domains but different C-terminal (C-t) domains of 36 and 57 amino acids respectively, due to a frameshift caused by the alternative splicing of intron I (FIG. 1b). For SIBRC1b, inventors isolated a single type of cDNA containing a 1089 bp long ORF split by one intron (FIG. 1b).

Alignment of the short and long cDNAs of SIBRC1a, the cDNA of SIBRC1b and the SIBRC1a and SIBRC1b genomic sequences confirmed that intron II of SIBRC1a was co-linear with the intron of SIBRC1b, suggesting that this intron was also spliced in the common ancestor, before duplication. In contrast, intron I of SIBRC1a could have evolved later, after duplication: it had frame conservation and a high sequence similarity to a coding (unspliced) region of SIBRC1b. Moreover, the C-t peptide encoded by the long SIBRC1a cDNA (which retained intron I) had sequence similarity to the C-t peptide of SIBRC1b, supporting that this frame was translated in the common ancestor, which did not splice this region. The putative splicing sites (gt/ag) of intron I were found in all the Solanum BRC1a genes studied but not in BRC1b genes, suggesting that they evolved within the BRC1a clade. Interestingly, the C-t region of the short BRC1a protein was predicted to form a strongly amphpathic a-helix (FIG. 3) highly conserved in all the Solanum species analyzed. On the other hand the C-t peptide of the long SIBRC1a protein was predicted to have an extended secondary structure (FIG. 3) like that of SIBRC1b. These results indicate that, after the separation of Arabidopsis and Solanaceae, a duplication of an ancestral BRC1-like gene, which had one intron, generated two gene copies, BRC1a and BRC1b. A new alternative splicing site evolved in the BRC1a Glade generating a divergent transcript, with a 3′-terminal frameshift, encoding a protein with a novel C-t motif predicted to form a strongly amphipathic α-helix.

Since the putative splicing sites (gt/ag) of intron I were found in all the Solanum BRC1a genes studied but not in BRC1b genes, inventors followed this pattern of alternative splicing of BRC1a gene in the genome sequence of SpBRC1a gene and, after was identified said intron I, inventors predicted the short cDNA coding for SEQ ID NO: 1 and the long cDNA coding for SEQ ID NO: 2.

### Example 2

### SpBRC1a Acts as a Suppressor of Shoot Branching in Tomato Role of SIBRC1a and SIBRC1b

The finding of two tomato BRC1-like paralogs more closely-related to each other than with AtBRC1, suggests that a duplication of BRC1 took place after the separation of the Brassicaceae and Solanaceae. These two BRC1-like genes map to different chromosomes, suggesting that they did not originate by segmental duplication but rather by chromosomal duplication or during the whole-genome duplication (WGD) occurred in Solanaceae 60-70 mya. Duplication, a common event in the evolution of genomes, is usually followed by loss of one of the gene copies by accumulation of deleterious mutations. In Arabidopsis, for instance, a single BRC1 gene has been found, although this species has undergone several WGD suggesting that all the other BRC1-like gene copies have been lost. A large number of models have been proposed to explain fixation, maintenance and evolution of duplicate genes.

These results indicate that in the case of tomato BRC1-like genes, SIBRC1b seems to have retained the ancestral BRC1-like gene function in the suppression of shoot branching while inventors have not been able to establish any relevant role in this process for SIBRC1a. Consistently, an asymmetrical distribution of selective constraints has been detected in the evolution of Solanum BRC1a and BRC1b lineages: the coding sequence of BRC1b genes have evolved under a strong purifying selection while the BRC1a lineage had experienced a decrease of evolutionary constraints.

### 2.1. Role of SIBRC1a and SIBRC1b in Tomato and its Evolution in Solanum Genus.

As far as it is known, SIBRC1b acts as a suppressor of shoot branching in tomato whereas SIBRC1a does not play major role in the control of shoot branching in tomato (WO 2010/081917 A1). In the above-mentioned patent application inventors arrived to this conclusion after carrying out experiments using RNAi for each of the genes SIBRC1a and SIBRC1b, taking this fact into account in the final claims, because neither the use of SIBRC1a nor the use of SIBRC1a-RNAi contributed to solve any technical problem. In the present application inventors repeated the experiments of silencing and the same results that it is shown in WO 2010/081917 were obtained; SIBRC1b loss-of function leads to increased branch outgrowth in tomato but SIBRC1a loss-of function does not cause bud outgrowth. As a result, these examples are excluded in the present application because they do not provide more relevant information.

2.1.1. Role of SIBRC1a

The Solanum lycopersicum wild-type branching patterns of strong Slbrc1a-v220i mutants could indicate that SIBRC1a does not play a major role in the suppression of axillary bud outgrowth in tomato, raising the possibility that this gene has become non-functional in this species. Other signs of pseudogenization such as the accumulation of missense mutations have not been found in this tomato gene. Moreover, transcription is tightly regulated spatially and temporally, and alternative splicing takes place, indicating that transcriptional and post-transcriptional regulation have not been lost. No signs of a trend towards pseudogenization have been detected in the evolution of the Solanum BRC1a lineage, either. These results indicate that the fast evolution rate found in this clade is not due to relaxation (usually associated with loss of function), but rather to positive selection and adaptation. Moreover, inventors have identified several protein sites, targets of positive selection and evolving at high evolution rate, located in domains potentially involved in stability (PEST domain) and transcriptional activity, aspects which could greatly affect protein function. In addition, the alternative splicing evolved within the BRC1a clade gives rise to a novel transcript (identified both in tomato and potato) encoding a divergent protein with a C-t amphipathic helix, secondary structure often involved in protein-protein interactions, dimerization and transcriptional activation or repression. Inventors speculate that this novel domain, generated by a 3′ terminal (3′-t) frameshift of the BRC1a transcripts, could also be contributing to the structural (and perhaps functional) divergence of BRC1a proteins. 3′-t frameshift mutations have been proposed to be responsible for the evolution of novel protein domains, instrumental to the diversification of transcription factor families. In summary, the BRC1a clade could be diversifying rather than leading to loss of function and pseudogenization.

In the particular case of the tomato SIBRC1a gene, its low transcription levels and the apparently irrelevant function could be result of the recent artificial evolution associated to tomato domestication. Under-expressor alleles could have been fixed during the selection of other linked loci controlling selected traits. Alternatively, SIBRC1a could be functional but only up-regulated under certain developmental or environmental conditions not yet identified. So far, inventors have not detected up-regulation of SIBRC1a in plants with reduced SIBRC1b function, for instance, but other genetic or environmental situations might lead to transcriptional activation of SIBRC1a.

2.1.2. Role of SIBRC1b

The excess of shoot branching displayed by SIBRC1b loss-of-function plants resembles that of tb1/brc1 mutants in other species (Aguilar-Martinez et al. 2007. Plant Cell, 19: 458-472; Doebley et al. 1997. Nature, 386: 485-488; Finlayson 2007. Plant Cell Physiol., 48: 667-677; Minakuchi et al. 2010. Plant Cell Physiol, 51: 1127-1135, Takeda et al. 2003. Plant J., 33: 513-520) and indicates that SIBRC1b must have retained the ancestral role in branch growth suppression. This is consistent with the low evolution rate (w) of the BRC1b genes in Solanum. SIBRC1b cis-regulatory regions and trans-acting factors also seem to be conserved: SIBRC1b is mainly expressed in arrested axillary buds, especially in basal nodes, like AtBRC1 and rice fine culm1, and it responds to decapitation like AtBRC1 (Aguilar-Martinez et al. 2007, Arite et al. 2007. Plant Journal, 51: 1019-1029). In Arabidopsis and rice it has been proposed that AtBRC1 and FC1, respectively, are downstream of the strigolactone pathway (Aguilar-Martinez et al. 2007. Plant Cell, 19: 458-472; Minakuchi et al. 2010. Plant Cell Physiol, 51: 1127-1135). Although in tomato this needs to be tested, loss of function of SICCD7, a gene involved in tomato strigolactone synthesis (Vogel et al. 2009. Plant J, 61, 300-311; Koltai et al. 2010. J Exp Bot, 61: 1739-1749) causes alterations in vegetative but not in sympodial branching, similarly to loss of SIBRC1b function. However, Slccd7 phenotypes are much stronger than those of SIBRC1b RNAi lines suggesting that additional factors may be controlled by this gene. Other related TCP genes such as SIBRC1a, SIBRC2a and SIBRC2b, are expressed in axillary buds at much lower levels than SIBRC1b but inventors cannot rule out that, in some conditions, they are relevant for this process.

### 2.2. Use of SpBRC1a as a Suppressor of Axillary Shoot Branching in Tomato

Different tomato seed stocks obtained from the Tomato Genetics Resource Centre (TGRC) at the University of California Davis were grown. Special mention can be done with respect to the introgression line IL3-5. As it is indicated in the “chromosomal mapping” section, mapping was done in a Solanum pennellii introgression population using primers combinations and, after detection of the corresponding PCR fragments, the inventors concluded that BRC1a mapped to IL3-5 (FIG. 2), in addition BRC1b mapped to IL6-2 and BRC2a to IL4-1. Therefore, the introgressed fragment from Solanum pennellii contains SpBRC1a gene. For these plants IL3-5, neither “branchless” phenotype nor the expression of SpBRC1a has been mentioned to date.

Interestingly, as shown in FIG. 4, IL3-5 lines, carrying a genomic region comprising a SpBRC1a gene, expressed this gene, SpBRC1a, at four times higher levels than SIBRC1a is expressed in M82 control plants and they display stronger apical dominance than M82 plants. These results demonstrate that introgression of expressed wild SpBRC1a genes into production tomato lines generates plants with improved architecture and reduced lateral shoot branching (FIGS. 4 and 5).

### Example 3

### SpBRC1a is the Responsible for the Strong Apical Dominance of Introgression Line IL3-5 Plants and Also of SpBRC1a Solanum lycopersicum Transgenic Plants

To confirm that SpBRC1a, contained in the introgression line IL3-5, is the gene responsible for the strong apical dominance of these lines, inventors caused silencing of SpBRC1a by generating RNAi transgenic lines carrying a sequence that is identical to part of its coding sequence. Inventors compared the branching phenotype of transgenic plants with those of M82 and with those carrying an empty binary vector. SpBRC1a was the responsible for the branch suppression RNAi lines having a similar number of branches as M82 plants.

### 3.1. SpBRC1a and the Strong Apical Dominance of Introgression Line 1L3-5 Plants.

3.1.1. CaMV35S:RNAi Constructs

A BRC1a-specific PCR product (225 bp) was cloned into the vector pHANNIBAL (CSIRO) using restriction sites BamHI/Clal and Xhol/Kpnl for the first and second cloning, respectively. Primers used were A and B:

(SEQ ID NO: 17)
A: 5′GGGGCTCGAGGGATCCTGGCTACTCACAAAGTCAAAATCAGCG
3′.
(SEQ ID NO: 18)
B: 5′GGGGGGTACCATCGATAACAACTGGATGAATTATTGCCCTACG
3′.

The pHANNIBAL cassettes were digested with NotI and subcloned in the NotI site of Bluescript SK+. The cassette was then digested with SacI/SmaI and cloned in the MCS of the binary vector pBIN19. For the controls inventors will use an empty pBIN19 vector.

3.1.2. Generation of IL3-5 Tomato Transgenic Plants Comprising the RNAi Constructs.

Binary vectors were transformed into Agrobacterium tumefaciens strain LBA4404. To generate stable transgenic tomato plants inventors will use the Solanum pennellii introgression line IL3-5 in the background of S. lycopersicum cv. M-82 (LA4060). Inventors transformed cotyledons according to (Ellul et al. 2003. Theor Appl Genet, 106: 231-238). Shoots regenerated from calli grown in kanamycin were transferred to non-selective root inducing media and were confirmed for transgene integration by PCR using primers Agri51 and

PDK_Hannibal_51.
Agri51:
(SEQ ID NO: 19)
5′CAACCACGTCTTCAAAGCAA 3′.
PDK_Hannibal_51:
(SEQ ID NO: 20)
5′ATCTTCTTCGTCTTACACATCACTTG 3′.

Levels of transgene expression were quantified by Real-Time Q-PCR in RNA from leaves. Ploidy level of transformants was analyzed in leaf tissue, using wild type tissue as diploid reference. Nuclei were isolated according to (Galbraith et al. 1983. Science, 220: 1049-1051). Cytometry analyses were performed using a flow cytometer Beckman Coulter EPICS XL-MCL. Branch lengths were measured in mm immediately after anthesis of the first flower

3.2. SpBRC1a Solanum lycopersicum Transgenic Plants Show Branchless Phenotype.

In addition, in order to confirm that SpBRC1a contributes to promote a stronger apical dominance than that of control plants, inventors generated transgenic lines carrying the genomic sequence of SpBRC1a; SEQ ID NO: 5. Inventors compared the branching phenotype of transgenic plants with those of M82 control plants and with those carrying an empty binary vector. Inventors observed that transgenic plants have fewer number of branches than M82 plants.

SpBRC1a was cloned in pBIN19 vector under the control of the SpBRC1a promoters. Binary vectors were transformed into Agrobacterium tumefaciens strain LBA4404 and transgenic plants of S. lycopersicum cv. M-82 were generated after regeneration.

### EXPERIMENTAL PROCEDURES

### Plant Material

Tomato seed stocks were obtained from the Tomato Genetics Resource Centre (TGRC) at the University of California Davis or kindly provided by R. Fernandez Muñoz (Estación Experimental La Mayora, Murcia, Spain). Accession numbers: Moneymaker (MM), LA2706; Solanum pennellii, LA0716; Solanum pimpinellifolium, PE-2; Solanum habrochaites, LA1777; Solanum galapagense, LA0317; Solanum arcanum, PE-30; Solanum lycopersicum, LA2706; Solanum habrochaites glabratum, PI 126449; Solanum huaylasense, LA2561; Solanum chilense (1969), LA1969; Solanum peruvianum, LA0098; Solanum chmielewskii, LA1028; Solanum corneliomulleri, LA0366; Solanum pennellii introgression lines in the background of S. lycopersicum cv. M-82, IL3-5, LA4047; LA4060. TILLING Slbrc1a point mutant alleles were obtained from tomato M82 EMS-mutagenized population and a Red Setter EMS-mutagenized population. Seeds were germinated in soil and transplanted to 1.5 L pots and grown in chambers at 21° C., long photoperiod (16 h light 8 hours dark), PAR 90 mmol m2 s-1.

### Identification and Isolation of Tomato BRC1-Like Genes

Inventors carried out BLAST searches at the TIGR Solanaceae Genomics Resource site (http://jcvi.org/potato/sol_ma_blast.shtml), TIGR Plant Transcript Assemblies Database (http://plantta.jcvi.org/) and SOL Genomics Network Tomato Gene Index website (http://solgenomics.net/) using the TCP domain of AtBRC1. To obtain full length cDNAs, RNA from dissected axillary buds was extracted with the guanidine-HCl method. cDNA was synthesized and full length cDNAs were isolated using primers indicated in Table S2.

### Chromosomal Mapping of the CYC/TB1-Like Genes

Genomic fragments of BRC1a, BRC1b and BRC2a were amplified from L. pennellii and sequenced to find PCR based markers. Mapping was done in an L. pennellii introgression population (Eshed and Zamir 1995. Genetics, 141: 1147-1162) using primer combinations TCP1-F2 (5′ CCTCATAAAAGGGAATCAAGGGA 3′, SEQ ID NO: 13)/Le3 (5′ ATTGAGAATGACTTGAAAGATAAAGATGAG 3′; SEQ ID NO: 14) and the restriction enzyme Csp6l to detect a CAPS for BRC1a and TCP3-F2 (5′CCTCATAAAAGGGAATCAAGGGA 3′; SEQ ID NO: 15)/TCP3-R2 (5′ TATTGAAAAATCGCGCACGTA 3′; SEQ ID NO: 16) and BspHI for BRC2a. A PCR-fragment length polymorphism for BRC1b was detected using primers TCP2-F2/LeTCP2 cDNA-R. BRC1a mapped to IL3-5, BRC1b to IL6-2 and BRC2a to IL4-1.

### Phenotypic Analysis

Branch lengths were measured in mm immediately after anthesis of the first flower.

### Real-Time PCR

Plant tissue was harvested and RNA was isolated with the guanidine-HCl method. Each biological replicate came from individually dissected axillary buds (Ax buds) from nodes 1-2, 3-4, 5-6 and 7-8 from 12-15 individuals. For the other tissues (seed, stem, leaf, floral organs) each replicate contained material from 7-9 individuals. Traces of DNA were eliminated with TURBO DNA-free© (Ambion). Two g of RNA were used to make cDNA with the High-Capacity cDNA Archive Kit (Applied Biosystems). Quantitative PCR was performed with SYBR® Green PCR Master Mix (Applied Biosystems) and the Applied Biosystems 7500 real-time PCR system, according to the manufacturer's instructions. Primers used are in Table S2. Three biological replicates were analyzed in each case. Ct values were obtained with the 7500 Systems SDS software 1.3.1.21 (Applied Biosystems). Relative fold expression changes were calculated as described in (Aguilar-Martinez et al. 2007. Plant Cell, 19: 458-472). ACTIN8 whose expression levels are constant in all the tissues and conditions analyzed was used as a normalizer. In all the figures the calibrator is the sample with the highest expression.

Isolation of BRC1-Like Genes from Wild Tomato Species

Young leaf tissue was collected in N(I), grinded and used to extract genomic DNA with the DNeasy Plant Mini kit (QIAGEN). To amplify the coding regions of BRC1a and BRC1b inventors carried out nested Pwo polymerase (ROCHE) PCR reactions according to manufacturer's instructions using primers indicated in Table S2.

### Phylogenetic Analyses

TCP domain-coding DNA sequences were aligned with MUSCLE (v3.7) and default parameters (Edgar 2004. Nucleic Acids Res, 32: 1792-1797). Test of best nucleotide substitution evolutionary model was done with MODELTEST. The best fit model (AIC selection) was K81+G+I (w parameter for gamma distribution=0.725). Maximum likelihood (ML) tree reconstruction with the best model and 1000 bootstrap pseudoreplicates was run in PhyML (v3.0 aLRT). The complete phylogenetic pipeline was run in Phylemon web server. Tree was represented with TreeDyn (v198.3).

1. An isolated polynucleotide, encoding a polypeptide comprising:
a. an amino acidic sequence from Solanum pennellii which is at least 95% identical over the full length of SEQ ID NO: 1 or of SEQ ID NO: 2, or
b. the amino acidic sequence from Solanum pennellii SEQ ID NO: 1 or SEQ ID NO: 2.

2. An isolated polynucleotide, encoding a polypeptide consisting of:
a. an amino acidic sequence from Solanum pennellii which is at least 95% identical over the full length of SEQ ID NO: 1 or of SEQ ID NO: 2, or
b. the amino acidic sequence from Solanum pennellii SEQ ID NO: 1 or SEQ ID NO: 2.

3. The polynucleotide according to claim 2, wherein said polynucleotide is
a. SEQ ID NO: 3 encoding SEQ ID NO: 1, or
b. SEQ ID NO: 4 encoding SEQ ID NO: 2.

4. The polynucleotide according to claim 2, wherein said polynucleotide is the genomic sequence SEQ ID NO: 5.

5-16. (canceled)

17. The polynucleotide according to claim 1 operatively linked to any regulatory sequence that controls the expression of said polynucleotide.

18. The polynucleotide according to claim 17, wherein the regulatory sequence is SEQ ID NO: 6, SEQ ID NO: 7 or CaMV35S.

19. An expression product of the polynucleotide according to claim 1.

20. An expression vector comprising the polynucleotide according to claim 1.

21. A host cell, having the polynucleotide according to claim 2 wherein if the host cell is a plant cell, said plant cell is not a Solanum pennellii cell.

22. A host cell, having the polynucleotide according to claim 2 operatively linked to any regulatory sequence that controls the expression of said polynucleotide wherein if the host cell is a plant cell, said plant cell is not a Solanum pennellii cell.

23. A host cell, having the expression product of the polynucleotide according to claim 2 wherein if the host cell is a plant cell, said plant cell is not a Solanum pennellii cell.

24. A host cell, having the expression vector comprising the polynucleotide according to claim 2 or comprising the polynucleotide thereof operatively linked to the regulatory sequence, wherein if the host cell is a plant cell, said plant cell is not a Solanum pennellii cell.

25. A tomato plant, but not of the species Solanum pennellii, having the polynucleotide according to claim 2, wherein said plant has fewer and/or shorter axillary shoots than a control plant.

26. A tomato plant, but not of the species Solanum pennellii, having the polynucleotide according to claim 2 operatively linked to any regulatory sequence that controls the expression of said polynucleotide, wherein said plant has fewer and/or shorter axillary shoots than a control plant.

27. A tomato plant, but not of the species Solanum pennellii, having the expression product of the polynucleotide according to claim 2, wherein said plant has fewer and/or shorter axillary shoots than a control plant.

28. A tomato plant, but not of the species Solanum pennellii, having the expression vector comprising the polynucleotide according to claim 2 or comprising the polynucleotide thereof operatively linked to the regulatory sequence, wherein said plant has fewer and/or shorter axillary shoots than a control plant.

29. A tomato plant, but not of the species Solanum pennellii, comprising the host cell according to claim 20, wherein said plant has fewer and/or shorter axillary shoots than a control plant.

30. The tomato plant according to claim 25, wherein said tomato plant is a Solanum lycopersicum plant.

31. The tomato plant according to claim 27, wherein the expression of the polynucleotide is greater than the expression of the homologous native gene BRC1a.

32. A germplasm of the tomato plant according to claim 25.

33. A method for producing tomato plants having fewer and/or shorter axillary shoots than a control plant, comprising:
a. Transforming at least a tomato plant cell with,
i. the polynucleotide according to claim 2, heterologously, and
b. growing the plant cell obtained in the step (a) in a suitable medium to produce at least a plant which expresses the heterologous polynucleotide.

34. The method according to claim 33, wherein the tomato plant is a Solarium lycopersicum plant.


SEQUENCE LISTING (20 sequences)

SEQ ID NO: 1 - protein, 313 aa, Solanum pennellii
MYPSSNNSPNISSSSSFFHINIPSPSMQYEPEFIQYFQDFQFIQPSYDQNNLDTNITREDSDKLDKIEEDQSIIKSCNNKKDEKSSSSTSTIRRKNNKRTTSGSAGVGPSKKDRHSKINTAHGPRDRRMRLSLEIARKFFNLQDLLGFDKASKTVEWLLTKSKSAVNDLVQKINKDKCSGSTNPNIATVSSPSESCEVISGVIDESAATNTAETHKQQKKKVKSIRRAIFHPVVAKESRKEARARARERTIIKKSLNDNTNNNNNGDQSMADEDLTRSLGSWNTTFEDHQSATGAHLCSTITRSILKFLKSIN

SEQ ID NO: 2 - protein, 349 aa, Solanum pennellii
MYPSSNNSPNISSSSSFFHINIPSPSMQYEPEFIQYFQDFQFIQPSYDQNNLDTNITREDSDKLDKIEEDQSIIKSCNNKKDEKSSSSTSTIRRKNNKRTTSGSAGVGPSKKDRHSKINTAHGPRDRRMRLSLEIARKFFNLQDLLGFDKASKTVEWLLTKSKSAVNDLVQKINKDKCSGSTNPNIATVSSPSESCEVISGVIDESAATNTAETHKQQKKKVKSIRRAIFHPVVAKESRKEARARARERTIIKKSLNDNTNNNNNGDQSMADEDLTRSLGSWNTTFEDHQSGSQGYNNNNNNMNVVDNFNLVDTSNWSPFMFNYHQINTEISQEHQLTNFQYSGKLWEA

SEQ ID NO: 3 - DNA, 942 bp, Solanum pennellii
atgtacccttcgagcaataacagccccaatatttccagctcttcatctttttttcacattaatattccatctccttctatgcaatatgaacccgaattcatccaatatttccaagattttcaattcatccaacctagttacgatcagaataatctcgataccaatattactagagaagattcggacaaactagataaaatagaagaagatcaatcaatcataaaaagctgcaataacaagaaggatgagaagagtagtagcagtactagtactattcgtagaaaaaacaacaagagaactacgagtggtagtgctggtgtaggaccttcgaagaaagatagacacagcaaaatcaacacggcacatggcccaagagaccgaagaatgagactatcacttgaaattgctcgcaaattcttcaatttgcaagacttgcttgggttcgataaagccagcaaaactgtagaatggctactcacaaagtcaaaatcagcggtgaacgatctggttcagaaaattaacaaagacaaatgcagcggtagtacaaatcctaatattgctactgtatcatctccttccgaatcatgtgaagttatctctggagtaatcgacgaatcagctgcaactaatacagcagaaactcacaagcaacagaagaaaaaggttaagtcgattcgtagggcaatatttcatcccgttgttgcaaaggaatcaaggaaagaagcaagagctagggcaagggaaagaacaataataaagaaaagcctaaatgataacacgaataataataataatggtgatcaatctatggctgatgaggatttaacaagatcattaggatcttggaatactacatttgaagatcatcaatcagcaactggagcccatttatgttcaactatcaccagatcaatactgaaatttctcaagagcatcaattga

SEQ ID NO: 4 - DNA, 1050 bp, Solanum pennellii
atgtacccttcgagcaataacagccccaatatttccagctcttcatctttttttcacattaatattccatctccttctatgcaatatgaacccgaattcatccaatatttccaagattttcaattcatccaacctagttacgatcagaataatctcgataccaatattactagagaagattcggacaaactagataaaatagaagaagatcaatcaatcataaaaagctgcaataacaagaaggatgagaagagtagtagcagtactagtactattcgtagaaaaaacaacaagagaactacgagtggtagtgctggtgtaggaccttcgaagaaagatagacacagcaaaatcaacacggcacatggcccaagagaccgaagaatgagactatcacttgaaattgctcgcaaattcttcaatttgcaagacttgcttgggttcgataaagccagcaaaactgtagaatggctactcacaaagtcaaaatcagcggtgaacgatctggttcagaaaattaacaaagacaaatgcagcggtagtacaaatcctaatattgctactgtatcatctccttccgaatcatgtgaagttatctctggagtaatcgacgaatcagctgcaactaatacagcagaaactcacaagcaacagaagaaaaaggttaagtcgattcgtagggcaatatttcatcccgttgttgcaaaggaatcaaggaaagaagcaagagctagggcaagggaaagaacaataataaagaaaagcctaaatgataacacgaataataataataatggtgatcaatctatggctgatgaggatttaacaagatcattaggatcttggaatactacatttgaagatcatcaatcaggtagtcaaggctataataataataataataatatgaatgttgttgataactttaatttggtggatactagcaactggagcccatttatgttcaactatcaccagatcaatactgaaatttctcaagagcatcaattgacgaacttccagtattctgggaagttatgggaagcttaa

SEQ ID NO: 5 - DNA, 1296 bp, Solanum pennellii
atgtacccttcgagcaataacagccccaatatttccagctcttcatctttttttcacattaatattccatctccttctatgcaatatgaacccgaattcatccaatatttccaagattttcaattcatccaacctagttacgatcagaataatctcgataccaatattactagagaagattcggacaaactagataaaatagaagaagatcaatcaatcataaaaagctgcaataacaagaaggatgagaagagtagtagcagtactagtactattcgtagaaaaaacaacaagagaactacgagtggtagtgctggtgtaggaccttcgaagaaagatagacacagcaaaatcaacacggcacatggcccaagagaccgaagaatgagactatcacttgaaattgctcgcaaattcttcaatttgcaagacttgcttgggttcgataaagccagcaaaactgtagaatggctactcacaaagtcaaaatcagcggtgaacgatctggttcagaaaattaacaaagacaaatgcagcggtagtacaaatcctaatattgctactgtatcatctccttccgaatcatgtgaagttatctctggagtaatcgacgaatcagctgcaactaatacagcagaaactcacaagcaacagaagaaaaaggttaagtcgattcgtagggcaatatttcatcccgttgttgcaaaggaatcaaggaaagaagcaagagctagggcaagggaaagaacaataataaagaaaagcctaaatgataacacgaataataataataatggtgatcaatctatggctgatgaggatttaacaagatcattaggatcttggaatactacatttgaagatcatcaatcaggtagtcaaggctataataataataataataatatgaatgttgttgataactttaatttggtggatactagcaactggagcccatttatgttcaactatcaccagatcaatactgaaatttctcaagaggtatgaattaatttaattagtgaattgtttttttaattattattaatccgatcgcaaagtatttatttgtattttttctgttgcagcatcaattgacgaacttccagtattctgggaagttatgggaagcttaattagggcaaggaagttcttgttgatttagtgcaacttcagaagaaggctatttggtcatgttggtaatgtaaaatgaatttgtctattttattattatgtgtattccatgaaaaaacaacttgtatttttcttataaataagctcatctttatctttcaa

SEQ ID NO: 6 - DNA, 1723 bp, Solanum lycopersicum
agtctgaacccctttcacctcaactgtgggaagcaggaagaattcaccaaaactttaataacatattcagtaaaaatttttataatgcgtctaagaaaagtaaaatgtacgtagaatttatcctgcctcgtaaaaataaagattgtatctaaaaaaaacctcgactcaaataatacatattaacaaaattacaaattaactatcactcaaccccaatatttacttcaagttgttagggatcacttaagggcctttctttttctcctttttttttttttttttggagaagatgaaggtgaaagagatggtggatgatggagctaggaaagaggagattgaagggtattttttttgtcaaagtatgtgtcagttgctatcacgtgaacttgaaactaaggggcaccattagagaagactttagctataatatacattcatttctataaaaaaaaatcacmacataaacatgcccttttttaacttagctttaatatatcttttaaatttgattatgcagaaatagatatttaaatttatataaaatttaaaaagtctatctaacaatgttgtgtcctacatatattatatctggtatgatatatatgtgttacttgtttaattttatataaatttaaatatttatttatgcaaattcaaaattaagagatataaatatcaagctaaatcgaagttcaatgaaatatatatatataattatgccaatataaaatcagtgtaactatacaacaagtactatagtgtcccctccactctttttttctcaaattccctttcatactttaaactcccacatgagctagctagagaagtcttttttttttttaaagattcgkggtgtttacatcaatttaaacatattttgactaatttcatagaatatttatcatctcttattaataacatgtgtcatattcataaatgaatagaaattactaaatacagtagtactycttttaatttttttctaataaaatttaaacgtgaaacctcatgattcctaattatccacttcagtaaccatcgactcacaccaaccctttggtgcaagcgaagccttctttatctttatagcagataggggtcctttgaaaagatggaagtacaattacacctctctttgtccctttgcaggtaataacataacatgacttttctttatcttcatctttctttctttgtcaacaagaacatacaccaccatgaatgtctctcccattagctaatatattccagctaactagcttaaatatatagtgctaatacytgcacgaacacaaaaatagccactaatatacacctatacctagctattattattattatcataattaagcacwcaccaagcaacatacatgtaaagccacatatttttaatcacctgtctttctcaaccaaaaagctatattatcatcattatattgaaaaaaaaattaaaaataaccacatatcctttcccactttctctatgtgctatctttgtattcaaaatttatatatccaagagaattatgaagagtctctctcaaaaaaagttttaattaatttataaccttttcttttttcctactttttgttgatgcagctaggtagctagattattaaaagtgtcaaactgaagaagctgatgtttgtggttatttcaacttcaatacaagtgtgctaggttgtccttatcaaccagtttctttttttttttttaaag

SEQ ID NO: 7 - DNA, 665 bp, Solanum lycopersicum
tcacatgaaggggcacgataacaagttgttcgtatccatccattcacttccaacaataccgctacgtaccactacctgcttccttcctatccccagtctttgtcaaacttcctttccctttccaattactttttctttaatagagatgtttgtttccttttcccttccccccatattcttcccttttttttttatctctctttcacaatagtagcaccatgcctgtagctttgatgcttagacgggcgcacgcgcacgcgcactcacacaactagaatagaatcactctctctatatattcatagttatcaaaactacttatcatataccaaaaaaaaccactgtcattctcaagcaaataatattttttttaaaaaagaagaactacatatatatatatagtactactactattttcatcatcactttggtcaatccatacagttctaagtagtcattgcttcctctgtcaaattactgtatacagtacattgaactagctaggggaaaattaatctactaactctaatttgtttgtttaattctcttcttattgcagctagatttgcctaattagcagaaaaaccaaaagctgtgttcatactgtctttctcaagatctagacccaccatatagaccgcctcaactacagctactccacaaga

SEQ ID NO: 8 - protein, 325 aa, Solanum lycopersicum
MYPSSNYSPNISSSSSFFHINIPSPSMQYEPEFIQYFHDFQFIQPSYDQNTNIPAEEAADSDKLDKIEEDQSIIKSCNNNKKDEKSSSSTSTIRRKNNKRTTSGSAGVGPSKKDRHSKINTAHGPRDRRMRLSLEIARKFFNLQDLLGFDKASKTVEWLLTKSKSAVNDLVQKINKDKCSGSENPNIATVSSPSAESCEVIDESAATNTAETQKQQKKKVKSIRRAIIHPVVAKESRKEARARARERTIIKKSLNDNTNNNNNGDQSMADEDLTRSLRSWNTTFEDHQSAIGAHLCSTITKSILKFLKSINLRTSSILGSYGKLN

SEQ ID NO: 9 - protein, 300 aa, Solanum tuberosum
MYPSSPNISSSSSFFHINIPSPSMQYEPEFIQYFHDFQFIQPAAYDQNNLDTNITAEEGDHKMEEDELIMKSCKNKKDESTSTTTTIRRKNNKRTTSGTGVGPSKKDRHSKINTAHGPRDRRMRLSLEIARKFFNLQDLLGFDKASKTVEWLLTKSKSAVNDLVQKISKGKCSASTNPNIGVVSSPSESCEVISGVIDESAATNNTHKQQKKKKSIRRAIFHPVVAKESRTEARARARERTKIKKSLNNNNGDQSMAPDEDLTRSLGSWSTTFEDHQSAIGAHLCSTITKSILKFLRSIN

SEQ ID NO: 10 - protein, 346 aa, Solanum lycopersicum
MYPSSNYSPNISSSSSFFHINIPSPSMQYEPEFIQYFHDFQFIQPSYDQNTNIPAEEAADSDKLDKIEEDQSIIKSCNNNKKDEKSSSSTSTIRRKNNKRTTSGSAGVGPSKKDRHSKINTAHGPRDRRMRLSLEIARKFFNLQDLLGFDKASKTVEWLLTKSKSAVNDLVQKINKDKCSGSENPNIATVSSPSAESCEVIDESAATNTAETQKQQKKKVKSIRRAIIHPVVAKESRKEARARARERTIIKKSLNDNTNNNNNGDQSMADEDLTRSLRSWNTTFEDHQSGIQGYNNNNNMNVVDNFNLVDTSNWSPFMFNYHQINTEISQEHQFANFQYSGKLWEA

SEQ ID NO: 11 - protein, 336 aa, Solanum tuberosum
MYPSSPNISSSSSFFHINIPSPSMQYEPEFIQYFHDFQFIQPAAYDQNNLDTNITAEEGDHKMEEDELIMKSCKNKKDESTSTTTTIRRKNNKRTTSGTGVGPSKKDRHSKINTAHGPRDRRMRLSLEIARKFFNLQDLLGFDKASKTVEWLLTKSKSAVNDLVQKISKGKCSASTNPNIGVVSSPSESCEVISGVIDESAATNNTHKQQKKKKSIRRAIFHPVVAKESRTEARARARERTKIKKSLNNNNGDQSMAPDEDLTRSLGSWSTTFEDHQSGIQAYNNTNNIMNAVDNFNLVDTSNWSPFMFNYHQINTEISQEHQLTNFQYPGKLWEA

SEQ ID NO: 12 - protein, 362 aa, Solanum pennellii
MYPPSNNNCNYSPILSSLICQNIPSSPCMQYEHELYFQSFNHDDQYYFQQQQLVPLIDDLSPHILADSCIETITKPSNCNHVLEGMEEGRGEKKGDDDVRSSRISISKNNKRSSKKDRHSKINTARGPRDRRMRLSLDAARKFFRLQDLLGFDKASKTVEWLLTQSDSAIEELVAAKGNDAQVAQQTSCNTPTTTTGIGAICASNSISESCEVISGTDETSSNDKNKETAQDEKKKRKKVVNAARRAVLEPLTKESRNQARARARERTKSKKMSQTGKSKAPANDLNPSGSRRPANKTCEEAGTHEELNFHQEKNSVDDCNFMVNGNWNPFTIFSYHEQYAGISNEHQLVTDLQFCGKLWEG

SEQ ID NO: 13 - DNA, 23 bp, Artificial Sequence
  TCP3-F2 forward primer of SlBRC2a from Solanum lycopersicum
cctcataaaagggaatcaaggga

SEQ ID NO: 14 - DNA, 30 bp, Artificial Sequence
  Le3 reverse primer of SlBRC1a from Solanum lycopersicum
attgagaatgacttgaaagataaagatgag

SEQ ID NO: 15 - DNA, 23 bp, Artificial Sequence
  TCP3-F2 forward primer of SlBRC2a from Solanum lycopersicum
cctcataaaagggaatcaaggga

SEQ ID NO: 16 - DNA, 21 bp, Artificial Sequence
  TCP3-R2 reverse primer of SlBRC2a from Solanum lycopersicum
tattgaaaaatcgcgcacgta

SEQ ID NO: 17 - DNA, 43 bp, Artificial Sequence
  A forward primer
ggggctcgagggatcctggctactcacaaagtcaaaatcagcg

SEQ ID NO: 18 - DNA, 43 bp, Artificial Sequence
  B reverse primer
ggggggtaccatcgataacaactggatgaattattgccctacg

SEQ ID NO: 19 - DNA, 20 bp, Artificial Sequence
  Agri51 forward primer
caaccacgtcttcaaagcaa

SEQ ID NO: 20 - DNA, 26 bp, Artificial Sequence
  PDK_Hannibal_51 reverse primer
atcttcttcgtcttacacatcacttg